SEQUENCE LISTING (4 sequences)

SEQ ID NO: 1 - DNA, 28 bp, artificial sequence
  sythetic primer
actcgagtcagcagaccggtactgcaga

SEQ ID NO: 2 - DNA, 28 bp, artificial sequence
  synthetic primer
gtcgactatttcagttcaaccgttgcac

SEQ ID NO: 3 - DNA, 2043 bp, artificial sequence
  synthetic construct
tcgactcagcaaaccggtactgcagaactgattatgaagcgggtgatgctggaccttaaaaagcctttgcgcaatatggataaggtggcggaaaagaacctgaatacgctgcagcctgacggtagttggaaggatgtgccttataaagatgatgccatgaccaattggttgccaaacaaccacctgctacaattggaaactattatacaggcttatattgaaaaagatagtcactattatggcgacgataaagtgtttgaccagatttccaaagcttttaagtattggtatgacagcgacccgaaaagccgcaactggtggcacaatgaaattgccactccgcaggcccttggtgaaatgctgatcctgatgcgttacggtaaaaagccgcttgatgaagcattggtgcataaattgaccgaaagaatgaagtggggcgaaccggagaagaaaacgggggccaacaaaacagatatcgccctgcattacttttatcgtgctttgttaacgtctgatgaggctttgctttccttcgccgtaaaagaattgttttatcccgtacagtttgtacactatgaggaaggcctgcaatacgattattcctacctgcagcacggtccgcaattacagatatcgagctacggtgccgtatttattaccggggtactgaaacttgccaattacgttagggataccccttatgctttaagtaccgagaaactggctatattttcaaagtattaccgcgacagttatctgaaagctatccgtggaagttatatggattttaacgtagaaggccgcggagtaagccggccagacattctaaataaaaaggcagaaaaaaagaggttgctggtggcgaagatgatcgatcttaagcatactgaagaatgggctgatgcgatagcctggacagatagcacagttgcggccggctataagattgagccctatcaccatcagttctggaatggtgattatgtgcaacatttaagacctgcctattcttttaatgttcgtatggtgagtaagcggacccgacgcagtgaatccggcaataaagaaaacctgctgggcaggtatttatctgatggggctactaacatacaattgcgcggaccagaatactataacattatgccggtatgggaatgggacaagattcctggcataaccagccgtgattatttaaccgacagacctttgacgaagctttggggagagcaggggagcaatgactttgcaggaggggtgtctgatggtgtatacggggccagtgcctacgcattggattacgatagcttacaggcaaagaaagcctggttcttttttgacaaagagattgtatgtcttggtgccggtatcaacagcaatgcccctgaaaacattaccactacccttaaccagagctggttaaatggcccggttataagtactgcaggtaaaaccggccggggtaaaataacaacgtttaaagcacagggacagttctggttgttgcacgatgcgattggttattactttcctgaaggggccaaccttagtctgagtacccagtcgcaaaaaggcaattggttccacatcaacaattcacattcaaaagatgaagtttctggtgatgtatttaagctttggatcaaccatggtgccaggccagaaaatgcgcagtatgcttatatcgttttgccgggaataaacaagccggaagaaattaaaaaatataatggaacggcaccgaaagtccttgccaataccaaccagctgcaggcagtttatcatcagcagttagatatggtacaggctatcttctatacagctggaaaattaagcgtagcgggcatagaaattgaaacagataagccatgtgcagtgctgatcaagcacatcaatggcaagcaggtaatttgggctgccgatccattgcaaaaagaaaagactgcagtgttgagcatcagggatttaaaaacaggaaaaacaaatcgggtaaaaattgattttccgcaacaggaatttgcaggtgcaacggttgaactgaaatag

SEQ ID NO: 4 - protein, 680 aa, artificial sequence
  synthetic construct
STQQTGTAELIMKRVMLDLKKPLRNMDKVAEKNLNTLQPDGSWKDVPYKDDAMTNWLPNNHLLQLETIIQAYIEKDSHYYGDDKVFDQISKAFKYWYDSDPKSRNWWHNEIATPQALGEMLILMRYGKKPLDEALVHKLTERMKWGEPEKKTGANKTDIALHYFYRALLTSDEALLSFAVKELFYPVQFVHYEEGLQYDYSYLQHGPQLQISSYGAVFITGVLKLANYVRDTPYALSTEKLAIFSKYYRDSYLKAIRGSYMDFNVEGRGVSRPDILNKKAEKKRLLVAKMIDLKHTEEWADAIAWTDSTVAAGYKIEPYHHQFWNGDYVQHLRPAYSFNVRMVSKRTRRSESGNKENLLGRYLSDGATNIQLRGPEYYNIMPVWEWDKIPGITSRDYLTDRPLTKLWGEQGSNDFAGGVSDGVYGASAYALDYDSLQAKKAWFFFDKEIVCLGAGINSNAPENITTTLNQSWLNGPVISTAGKTGRGKITTFKAQGQFWLLHDAIGYYFPEGANLSLSTQSQKGNWFHINNSHSKDEVSGDVFKLWINHGARPENAQYAYIVLPGINKPEEIKKYNGTAPKVLANTNQLQAVYHQQLDMVQAIFYTAGKLSVAGIEIETDKPCAVLIKHINGKQVIWAADPLQKEKTAVLSIRDLKTGKTNRVKIDFPQQEFAGATVELK

(19) United States
(12) Patent Application Publication
(10) Pub. No.: US 2009/0286289 A1
(43) Pub. Date: Nov. 19, 2009

(54) Production of Hyaluronate Unsaturated Disaccharides and its Application

(76) Inventor: Danny Z. Pang, Fullerton, CA (US)

Correspondence Address: BIOTECH BEACH LAW GROUP , PC, 625 BROADWAY, Suite 1210, SAN DIEGO, CA, 92101, US

(21) Appl. No.: 12/399,943

(22) Filed: Mar. 7, 2009

Related U.S. Application Data
(60) Provisional application No. 61/035,316, filed on Mar. 10, 2008.

(51) Int. Cl. C12P 19/12; C12N 1/21

(52) U.S. Cl. 435/100; 435/252.35

(57) ABSTRACT

The present invention includes cell based systems and methods for production of hyaluronate unsaturated disaccharide. The methods and systems include the use of a host cell that produces hyaluronic acid transformed with an expression vector encoding a recombinant chondroitinase AC. Expression of chondroitinase AC in the presence of native hyaluronic acid results in digestion of hyaluronic acid into multiple units of hyaluronate unsaturated disaccharide, which can then be isolated.

### CROSS-REFERENCE TO RELATED APPLICATIONS

The present invention claims benefit of priority to U.S. patent application Ser. No. 61/035,316, filed on Mar. 10, 2008, which is herein incorporated by reference in its entirety.

### TECHNICAL FIELD OF THE INVENTION

The present application relates to the production of hyaluronate unsaturated disaccharides and more particularly to the use of a host cell expressing a recombinant chondroitinase AC in the presence of an endogenous hyaluronic acid to produce hyaluronate unsaturated disaccharide.

### BACKGROUND OF THE INVENTION

Hyaluronic acid (HA) is a naturally occurring non-sulfated linear polyanionic, heteropolysaccharide with a repeating disaccharide unit consisting of D-glucuronic acid (1-3)-D-N-acetyl-glucosamine (1-4) with a molecular weight (MW) in the range 10⁴ to 10⁷ Dalton. HA has been used in many areas, such as ophthalmology, orthopedics, wound healing, drug delivery, coatings, implants and therapeutics. HA is the only glycosaminoglycan synthesized by both mammalian and bacterial cells particularly Lancefield groups A & C Streptococci. HA isolated and purified from both sources is structurally and chemically identical. Although most HA has been traditionally produced by extraction from animal tissues, a commercial or industrial production of HA has been steadily increased in recent years through fermentation of Group C streptococci, such as S. equi subsp. Zooepidemicus.

Chondroitinase AC or ABC selectively cleaves 1-4 glycoside bonds of HA and completely digests HA into an unsaturated disaccharide, known as hyaluronate unsaturated disaccharide (ΔDi-HA). ΔDi-HA has a structure of D-glucuronic acid (GlcUA) (1-3)-D-N-acetyl-glucosamine (GlcNAc) with a MW 378.31. In comparison to its parent, ΔDi-HA is more than 1,000 times less in MW, more stable in heat and moisture more resistant to enzymatic and chemical degradation, and less allergenic. ΔDi-HA is the basic building block for glycosaminoglycan in many organs and tissues. ΔDi-HA has the basic structure of glucosamine and chondroitin.

Currently, ΔDi-HA is only available as a reagent or a reference material, mainly due to a high cost of production of ΔDi-HA by the enzymatic digestion of pure HA (substrate) with chondroitinases. Thus, generation of a ΔDi-HA reagent involves the in vitro incubation of substrate and enzyme under suitable conditions. Production therefore requires purification of the active enzyme, substrate and the in vitro culturing together for proper digestion. Due to the high cost and the extreme limit supply of ΔDi-HA, the functions of ΔDi-HA are hardly explored. Therefore there remains a need to develop improved systems and methods for the production of ΔDi-HA.

### BRIEF SUMMARY OF THE INVENTION

The present invention addresses the deficiencies found in current production methods of hyaluronate unsaturated disaccharides and provides related benefits. Specifically the present invention includes methods of producing hyaluronate unsaturated disaccharides that eliminates the requirement of purification of a functional protein, the purification of hyaluronic acid and maintaining suitable in vitro conditions for enzymatic digestion. More specifically the present invention advances current methodologies used in the production of hyaluronate unsaturated disaccharide by providing a cell based system that efficiently produces hyaluronate unsaturated disaccharide.

In one aspect of the present invention a method of producing a hyaluronate unsaturated disaccharide is provided, the method including providing a host cell capable of expressing a native or endogenous hyaluronic acid, the host cell including a recombinant expression vector including a nucleic acid sequence encoding a chondroitinase AC polypeptide operably linked to a promoter, culturing the host cell in conditions suitable for expression of the chondroitinase AC polypeptide and for a time sufficient for the digestion of hyaluronic acid into hyaluronate unsaturated disaccharide by the expressed chondroitinase AC polypeptide, and isolating the hyaluronate unsaturated disaccharide. The host cell does not express a native or endogenous chondroitinase AC polypeptide.

In another aspect of the present invention a method of producing a hyaluronate unsaturated disaccharide is provided the method including providing a host cell capable of expressing a native or endogenous hyaluronic acid, the host cell including a recombinant expression vector including a nucleic acid sequence encoding a chondroitinase AC polypeptide operably linked to a promoter, inducing expression of the chondroitinase AC polypeptide, incubating the host cell in conditions suitable for digestion of hyaluronic acid into hyaluronate unsaturated disaccharide by the chondroitinase AC polypeptide and isolating the hyaluronate unsaturated disaccharide.

In another aspect of the present of the present invention a method of producing a hyaluronate unsaturated disaccharide is provided the method including providing a host cell capable of expressing a native or endogenous hyaluronic acid, constructing an expression vector including a nucleic acid sequence encoding a chondroitinase AC polypeptide operably linked to a promoter transforming or transducing the host cell with the expression vector, inducing expression of the chondroitinase AC, incubating the host cell in conditions suitable for cleavage of hyaluronic acid by chondroitinase AC into hyaluronate unsaturated disaccharide and isolating the hyaluronate unsaturated disaccharide.

In another aspect of the present invention a host cell capable of producing hyaluronate unsaturated disaccharide is provided, the host cell capable of expressing native or endogenous hyaluronic acid transfected or transformed with a recombinant expression vector comprising a nucleic acid sequence encoding a foreign or exogenous chondroitinase AC polypeptide operably linked to a promoter.

### BRIEF DESCRIPTION OF THE DRAWINGS

FIG. 1 is a schematic representation depicting the construction of an expression vector pGK/AC, which encodes a chondroitinase AC polypeptide. More specifically, a nucleic acid sequence encoding a chondroitinase AC polypeptide was selectively amplified by PCR using Taq polymerase and the primers provided in SEQ ID NOS: 1 and 2. The amplified fragment was ligated to a cloning vector including T overhangs. The insert was then restricted using restriction enzymes and subsequently subcloned into a plasmid having complementary restriction sites. The insert was further shuttled through subsequent restriction and ligation procedures into a pGK:nucMCS expression vector designated pGK/AC.

FIG. 2 is a photograph of beta-hemolysis as white sport (clear zone) or rigns around S. Zooepidemicus colonies transformed with the pGK/AC expression vector. Cells were electroporated with the pGK/AC construct and cultured at 37 degrees Celsius for 38 hours in media containing TSA/SB/Em^{R}. Colony with beta-hemolysis in media containing TSA/SB/Em^{R} demonstrates a success of transformation.

FIG. 3 is a photograph of beta-hemolysis around the sub-culture of pGK/AC/Zooepidemicus after 16 hours at 37 degrees Celsius. Control wells C1 and C2 include S. equi ss zooepidemicus without transformation, which shows beta hemolysis activity on TSA/SB but does not grow in TSA/SB/Em^{R} media. Black dots demonstrate the placement of these control cells.

FIG. 4 is a picture of Nobel-HA Plate Assay demonstrating the production of functional recombinant chondroitinase AC by transformed cells. Supernatant from cultures transformed with an expression vector including a recombinant chondroitinase AC were loaded into agar plates incorporating hyaluronic acid as substrate. Zones of clearing around wells 3S (pH 6.2) or ES (pH 8.2) verify chondroitinase AC enzymatic activity and thus demonstrate expression of a functional chondroitinase AC enzyme.

FIGS. 5A-B are monographs of HPLC (High-performance Liquid Chromatography), which depict detection peaks corresponding to hyaluronate unsaturated disaccharide. FIG. 5A depicts a control used as reference to demonstrate hyaluronate unsaturated disaccharide has a peak at about 4.950. FIG. 5B indicates the experimental reading from the isolated hyaluronate unsaturated disaccharide was 5.033. The isolated hyalunate unsaturated disaccharide was obtained after induction of clone pGK/AC/Zooepidemicus.

### DETAILED DESCRIPTION OF THE INVENTION

The present invention addresses deficiencies found in current production methods of hyaluronate unsaturated disaccharides and provides related benefits. Specifically the present invention includes methods of producing hyaluronate unsaturated disaccharides that eliminate the requirement of purification of a functional protein, the purification of hyaluronic acid and maintaining suitable in vitro conditions for enzymatic activity. The present invention advances current methodologies used in the production of hyaluronate disaccharide by providing a cell based system. More specifically the present invention includes the production of hyaluronate unsaturated disaccharide by controlled expression of a recombinant chondroitinase AC polypeptide in a transformed host cell. Thus the present invention includes the transformation of a host cell with an expression vector including a nucleic acid sequence encoding a chondroitinase AC polypeptide, which upon transcription then translation cleaves hyaluronic acid into the resulting hyaluronate unsaturated disaccharide. The present invention also includes a host cell transformed with an expression vector including chondroitinase AC and its application.

Thus it is an object of the present invention to provide a method of producing hyaluronate unsaturated disaccharide (ΔDi-HA) efficiently, inexpensively and at large quantity by using Group C streptococci as a production tool. It is another object of the present invention to produce hyaluronate unsaturated disaccharide in an amount equal or greater than 1 microgram. In another embodiment the present invention may be used to produce hyaluronate unsaturated disaccharide in an amount greater than 1 milligram. In another embodiment the present invention may be used to produce hyaluronate unsaturated disaccharide in an amount greater than 100 milligrams. In still other embodiments, one gram ten grams or more of hyaluronate unsaturated disaccharide are produced. In another embodiment of the present invention, the cell based production system is capable of producing at least 1 μg/mL of hyaluronate unsaturated disaccharide. In another embodiment the cell based system produces at least 0.1 mg/mL of hyaluronate unsaturated disaccharide. In another embodiment the cell based system produces at least 1 mg/mL of hyaluronate unsaturated disaccharide. In further embodiments the cell based system produces at least 10 mg/mL, 50 mg/mL, 100 mg/mL or more. Concentrations may be increased by decreasing the volume in which the final hyaluronate unsaturated disaccharide is provided.

In one aspect of the present invention a method of producing a hyaluronate unsaturated disaccharide is provided the method including providing a host cell capable of expressing a native or endogenous hyaluronic acid, the host cell including a recombinant expression vector including a nucleic acid sequence encoding a chondroitinase AC polypeptide operably linked to a promoter, culturing the host cell in conditions suitable for expression of the chondroitinase AC polypeptide and for a time sufficient for the digestion of hyaluronic acid into hyaluronate unsaturated disaccharide by the expressed chondroitinase AC polypeptide, and isolating the hyaluronate unsaturated disaccharide. In the preferred embodiment the host cell does not express a native or endogenous chondroitinase AC polypeptide.

In another aspect of the present invention a method of producing a hyaluronate unsaturated disaccharide is provided, the method including providing a host cell capable of expressing a native or endogenous hyaluronic acid, the host cell including a recombinant expression vector including a nucleic acid sequence encoding a chondroitinase AC polypeptide operably linked to a promoter, inducing expression of the chondroitinase AC polypeptide, incubating the host cell in conditions suitable for digestion of hyaluronic acid into hyaluronate unsaturated disaccharide by the chondroitinase AC polypeptide, and isolating the hyaluronate unsaturated disaccharide.

In another aspect of the present of the present invention a method of producing a hyaluronate unsaturated disaccharide is provided, the method including providing a host cell capable of expressing a native or endogenous hyaluronic acid, constructing an expression vector including a nucleic acid sequence encoding a chondroitinase AC polypeptide operably linked to a promoter, transforming or transducing the host cell with the expression vector, inducing expression of the chondroitinase AC, incubating the host cell in conditions suitable for cleavage of hyaluronic acid by chondroitinase AC into hyaluronate unsaturated disaccharide and isolating the hyaluronate unsaturated disaccharide.

In another aspect of the present invention a host cell capable of producing hyaluronate unsaturated disaccharide is provided, the host cell capable of expressing a native or endogenous hyaluronic acid transfected or transformed with a recombinant expression vector comprising a nucleic acid sequence encoding a foreign or exogenous chondroitinase AC polypeptide operably linked to a promoter.

In the preferred embodiment, the present invention utilizes host cells capable of expressing a native or endogenous hyaluronic acid but that do not express an endogenous, native or functional chondroitinase AC protein. Thus, a nucleic acid sequence encoding chondroitinase AC is introduced into the host cell by cell transformation with a suitable expression vector, then expressing chondroitinase AC by transcription followed by translation. In another embodiment, the present invention includes a host cell that expresses a dysfunctional chondroitinase AC polypeptide or a chondroitinase AC polypeptide at a very low level, or at level that is not readily measurable using conventional measuring techniques. Examples of host cells that may be used with the present invention include but are not limited to Group A and Group C streptococci. Group C streptococci are beta-hemolytic bacteria that occasionally cause human infections but more often are capable of causing infections in animals. Group A and C streptococci produce an endogenous hyaluronic acid. In the preferred embodiment of the present invention the Group C. streptococci is S. equi ss zooepidemicus. Host cells such as S. equi ss zooepidemicus may be obtained by tissue culture collections, such as but not limited to American Type Tissue Connection ((ATCC#35246) Manassas, Va.).

One skilled in the art would appreciate that since host cells are transformed with an expression vector, the cells may be made competent for the particular transformation process utilized. Once transformed host cells may be cultured in a selection media for selection of properly transformed cells. Examples include culturing host cells in media containing antibiotic to select for host cells capable to express an antibiotic resistance gene or sequence from the expression vector.

Host cells of the present invention are transformed with an expression vector including a nucleic acid sequence encoding a chondroitinase AC polypeptide or a functional portion thereof operably linked to a promoter. Thus the expression vector is used as a vehicle for the expression of chondroitinase AC. An expression vector is generally a plasmid that is used to introduce and express a specific gene into a target cell. Once inside and upon induction, the host cell transcribes the nucleic acid sequence into mRNA and translates the mRNA into a functioning polypeptide, protein or active fragment thereof. More specifically, the expression vector is capable of replication and capable of expression of an inserted nucleic acid sequence. Thus the expression vector includes an origin of replication for replication independent of the host cells chromosome or endogenous DNA and a promoter, which is optionally inducible by exposure to external stimuli. Inducible promoters are those under control by the user, typically by administering an activator or a compound that inhibits a repressor or by change in temperature. The expression vector may also have a selectable marker such as an antibiotic resistance gene or sequence and may have a terminator sequence for terminating transcription or translation.

Production of an expression vector including chondroitinase AC may include obtaining a nucleic acid sequence encoding chondroitinase AC and ligating the nucleic acid sequence with a vector or plasmid including a promoter suitable for expression. Ligation should occur such that a functional chondroitinase AC polypeptide or polypeptide fragment is expressed. Thus, ligation should occur such that the reading frame of the nucleic acid sequence is operably aligned with the promoter. In another embodiment, the expression vector is constructed by insertion of one or more promoters into a vector in addition to insertion of the nucleic acid sequence encoding chondroitinase AC.

In some embodiments, a nucleic acid sequence encoding chondroitinase AC is obtained by selective amplification from a cDNA library such as by using polymerase chain reaction (PCR). In another embodiment, the nucleic acid sequence encoding chondroitinase AC is obtained by PCR amplification of genomic DNA. In another embodiment, the nucleic acid sequence encoding chondroitinase AC is obtained by PCR amplification of a cDNA molecule. In another embodiment, the nucleic acid sequence encoding chondroitinase AC is obtained by isolating mRNA from a cell that expresses chondroitinase AC, reverse transcribing the mRNA, then selective amplification of the resulting cDNA. Thus in many of the disclosed embodiments, PCR may be used to amplify or generate a cDNA nucleic acid sequence encoding chondroitinase AC or an active fragment thereof. A variety of PCR methods may be adapted for use with the present invention by substituting primers indicated in such methods with a set of appropriate primers complementary to the nucleic acid sequence to be amplified, such as a nucleic acid sequence encoding chondroitinase AC. Among the PCR techniques included within the scope of the present invention include but are not limited to those described in U.S. Pat. Nos. 4,683,195, 4,683,202 and 4,800,159; each of which is herein incorporated by reference. Further or supplemental examples of PCR including RT-PCR as well as molecular cloning, subcloning and nucleic acid isolation or extraction techniques may be found in Sambrook et al. Molecular Cloning: A Laboratory Manual, 2d Ed., Cold Spring Harbor Laboratory Press, Cold Spring Harbor, N.Y., 1989, which is herein incorporated by reference.

In PCR amplification, a target nucleic acid sequence is amplified exponentially from a nucleic acid sequence or collection of nucleic acid sequences (such as a cDNA library) referred to as a template. More specifically synthetic primers such as sense and antisense primers are designed complimentary to the ends of a double stranded cDNA or gDNA sequence to be amplified and combined with the nucleic acid template (such as a nucleic acid sequence or library that encodes chondroitinase AC). DNA polymerase (such as Taq polymerase, Pfu polymerase), deoxynucleotide triphosphates (dNTPs), a buffer solution (providing a suitable chemical environment for activity and stability of the DNA polymerase), and divalent or monovalent cations to amplify a nucleic acid sequence encoding chondroitinase AC. The mixture is placed in a thermocycler, which typically includes a series of 20 to 35 repeated temperature changes called cycles; each cycle typically including 2-3 discrete temperature steps. Most commonly PCR is carried out with cycles that have three temperature steps. The cycling is often preceded by a single temperature step (called a hold) at a high temperature (>90° C.), and followed by one hold at the end for final product extension or brief storage. The temperatures used and the length of time they are applied in each cycle can depend on a variety of parameters. These include but are not limited to the enzyme used for DNA synthesis, the concentration of divalent ions and dNTPs in the reaction, and the melting temperature (Tm) of the primers.

In one embodiment of the present invention, the template or nucleic acid sequence used for amplification and thus production of cDNA encoding a chondroitinase AC polypeptide is genomic DNA (gDNA), which encodes chondroitinase AC. Genomic DNA is the full complement of DNA contained in the genome of a cell or organism. Genomic DNA is located in the cell nucleus of eukaryotes, as well as small amounts in mitochondria and chloroplasts. In prokaryotes, the genomic DNA is held within an irregularly shaped body in the cytoplasm called the nucleoid. Isolation of genomic DNA from a cell may be performed using genomic DNA extraction kits such as those provided by Invitrogen (Carlsbad, Calif.) and Promega (Madison, Wis.). Genomic DNA may be full length genomic DNA or may be sheared or partial nucleic acid sequences obtained from full length genomic DNA.

In another embodiment of the present invention the template or nucleic acid sequence used for amplification is cDNA or a cDNA library, which is a collection of multiple cDNAs. Complementary DNA or cDNA can be synthesized from a mature mRNA template in a reaction catalyzed by the enzyme reverse transcriptase. Methods for isolating mRNA from cells and reverse transcribing mRNA are well known in the art. Frequently these methods include lysing cells that produce the polypeptide of interest, isolating mRNA, such as by binding the poly A tail to solid phase then elution, reverse transcribing the mRNA, and amplifying the desired nucleic acid sequence using PCR with synthetic primers directed towards the desired nucleic acid sequence. Kits for such isolation, including reverse transcription may be obtained from a variety of companies such as Invitrogen (Carlsbad, Calif.). Primers should be designed specific to the target nucleic acid sequence and are explored in more depth below.

In another embodiment of the present invention the nucleic acid sequence includes a chondroitinase AC sequence according to SEQ ID NO:3. In another embodiment of the present invention, the nucleic acid sequence encodes the chondroitinase AC amino acid sequence provided in SEQ ID NO: 4. In another embodiment, the nucleic acid sequence encodes at least a portion of an active chondroitinase AC enzyme. Thus, the degeneracy of the genetic code encoding a chondroitinase AC polypeptide or an active fragment thereof is also to be interpreted as within the scope of the present invention. The genetic code has redundancy but no ambiguity. For example, although the codons GAA and GAG both specify glutamic acid (redundancy), neither of them specifies any other amino acid (no ambiguity). The codons encoding one amino acid may differ in any of their three positions. For example the amino acid glutamic acid is specified by GAA and GAG codons (difference in the third position), the amino acid leucine is specified by UUA, UUG. CUU, CUC, CUA. CUG codons (difference in the first or third position), while the amino acid serine is specified by UCA, UCG, UCC, UCU, AGU, AGC (difference in the first, second or third position). One skilled in the art may utilize a number of biochemistry or molecular biology text resources to identify appropriate substitutions or variations in the nucleic acid sequence that produce a functional polypeptide, protein or protein fragment.

Amplification of a nucleic acid sequence such as genomic DNA or cDNA encoding chondroitinase AC may be performed using polymerase chain reaction (PCR). The principles of PCR have been described above. Among the elements of PCR include the addition of synthetic primers to target amplification. Thus, synthetic primers should be designed according to the desired amplification; however, synthetic primer design also takes into account other factors. Among these include the melting temperature (Tm). The melting temperature of a synthetic primer is defined as the temperature at which 50% of that same DNA molecule species form a stable double helix and the other 50% have been separated to single strand molecules. The melting temperature required increases with the length of the primer. Primers that are too short would anneal at several positions on a long DNA template, which would result in non-specific copies. On the other hand the length of a primer is limited by the temperature required to melt it. Melting temperatures that are too high, i.e., above 80° C. can also cause problems since the DNA polymerases used for PCR are less active at such temperatures. The optimum length of a primer is generally from 20 to 30 nucleotides with a melting temperature between about 55° C. and 65° C. Pairs of primers should have similar melting temperatures as annealing in a PCR reaction occurs for both simultaneously. A primer with a Tm significantly higher than the reaction's annealing temperature may mishybridize and extend at an incorrect location along the DNA sequence, while Tm significantly lower than the annealing temperature may fail to anneal and extend at all. In addition, primer sequences need to be chosen to uniquely select for a region of DNA avoiding the possibility of mishybridization to a similar sequence nearby. Mononucleotide repeats should be avoided, as loop formation can occur and contribute to mishybridization. Primers should not easily anneal with other primers in the mixture (either other copies of same or the reverse direction primer); this phenomenon can lead to the production of primer dimer products contaminating the mixture. Primers should also not anneal strongly to themselves, as internal hairpins and loops could hinder the annealing with the template DNA.

In addition, synthetic primers may also be designed to incorporate one or more restriction sites into the amplified sequence. Such techniques may include substitutions or additions in the resulting nucleic acid sequence. Restriction sites, also referred to as restriction recognition sites, are specific sequences of nucleotides that are recognized by restricting enzymes. The sites are generally palindromic, (because restriction enzymes usually bind as homodimers) and a particular enzyme may cut between two nucleotides within its recognition site, or somewhere nearby. The incorporation of restriction sites may be chosen according to the particular expression vector. That is, restriction sites may be generated in a sequence to correspond with the same or similar restriction site found in the expression vector for desired insertion. A listing of restriction sites with corresponding restriction enzymes may be obtained by various molecular biology reagent suppliers such as New England Biolabs (Ipswich, Mass.). In addition synthetic primers may be utilized to incorporate multiple restriction sites to allow for the insertion of the nucleic acid sequence encoding chondroitinase AC in a variety of vectors or in a variety of reading frames. In some embodiments of the present invention, the synthetic primer sequences found in SEQ ID NO: 1 and SEQ ID NO:2 are used to amplify a nucleic acid sequence encoding chondroitinase AC. Synthetic primers may be ordered from a variety of molecular biology reagent suppliers and may be designed or further optimized using various software programs.

In one embodiment of the present invention, a template nucleic acid sequence encoding chondroitinase AC is PCR amplified using Taq polymerase which adds a single, 3′-A overhang to each end of the PCR product. This makes it possible to clone the PCR product directly into a linearized cloning vector with single, 3′-T overhangs. Typically, the PCR products with “A” overhang, are mixed with the vector in high proportion. Thus the complementary overhangs of “T” vector and PCR product can be ligated under the action of a ligase, such as T4 DNA ligase (New England Biolabs. Ipswich, Mass.). Vectors including “T” overhangs are commercially available from suppliers such as Invitrogen (Carlsbad, Calif.) and Promega (Madison, Wis.). These cloning vectors frequently include multiple restriction sites which allow the cloned insert to be removed and inserted into additional vectors including the same or similar restriction sites. Thus, once inserted into the cloning vector, the nucleic acid sequence encoding chondroitinase AC can be transcribed or can be removed by digestion with an appropriate restriction enzyme then ligated into the desired expression vector via complementary overhangs or blunt ends and in the presence of a ligase, such as T4 DNA ligase (New England Biolabs, Ipswich, Mass.).

Proper expression of chondroitinase AC or an active fragment thereof within a host cell requires the nucleic acid sequence be operably aligned with a promoter. A promoter is a regulatory region of DNA typically located upstream (towards the 5′ region) of a gene or nucleic acid sequence to be transcribed, which in essence provides a control point for the regulation of gene transcription. A promoter contains specific DNA sequences that are recognized by proteins known as transcription factors. These factors bind to the promoter sequences, recruiting RNA polymerase, the enzyme that synthesizes RNA from the DNA coding region. In the preferred embodiment, the promoter used to express recombinant chondroitinase AC is exogenous or not naturally occurring by the cell. Thus an exogenous promoter allows greater control in expression, whether inducing or repressing. Identifying a suitable promoter may be performed by inserting the promoter into an expression vector including a chondroitinase AC, transforming a suitable cell and inducing expression. If hyaluronic acid is cleaved into its unsaturated disaccharides, the promoter may be used with the present invention. In the most preferred embodiment, the promoter is the PGK promoter, however a variety of promoters are well known for use with expression vectors and thus the particular promoter is intended to be nonlimiting. For example, many promoters are adapted for recombinant expression systems such as but not limited to human cytomegalovirus (CMV), SV40 early promoter and the like. Multiple promoters may be identified through companies that produce expression systems such as Invitrogen (Carlsbad, Calif.).

In preferred embodiments an inducible promoter is provided within the expression vector, which allows the user to selectively induce or suppress expression. Specifically, an inducible promoter is one that in response to either the presence or absence of a particular compound or defined external condition, such as elevated temperature, the activity of the promoter and thus transcription can be controlled by the user. In some embodiments, expression of chondroitinase AC is modulated such as by selectively increasing or decreasing expression via an inducible promoter. Expression of chondroitinase AC may be selectively increased to cleave hyaluronic acid thus producing hyaluronate unsaturated disaccharide then selectively decreased to prevent or reduce cell toxicity or adverse cellular conditions. Expression of chondroitinase AC may also result in cleavage of tetra- and/or hexa-saccharides into hyaluronate unsaturated disaccharides. Expression vectors including those with inducible promoters are available from a variety of commercial suppliers. For example, Invitrogen (Carlsbad, Calif.) produces a variety of expression vectors that may be used with the present invention. Inducible promoters are commercially available that can be modulated by the presence or absence of IPTG, glucose, steroids, copper (cmt) and the like. The particular promoter is intended to be nonlimiting; however an exogenous promoter is preferred.

Insertion of the amplified nucleic acid encoding chondroitinase AC into an expression vector typically involves the process referred to in the molecular biology arts as ligation. Ligation is the joining of nucleic acid sequences using the enzyme lipase, such as T4 DNA lipase (New England Biolabs, Ipswich, Mass.). Ligation may involve the joining of blunt ends or sticky ends as is known in the molecular biology arts. Ligation may be unidirectional or bidirectional depending on the desires of the user. Moreover, a nucleic acid sequence may be ligated into a first vector, which includes a multicloning site (MCS), then digested with a restriction enzyme and ligated into a second vector and the like. In the preferred embodiment the directional insertion of the amplified nucleic acid sequence is performed by utilizing restriction sites incorporated into the ends of the nucleic acid sequence that encodes a functional chondroitinase AC protein or fragment thereof. Directional insertion into a vector can be controlled through the use of a multi cloning site (MCS) within an expression vector. Selection markers may be used to determine whether ligation of the nucleic acid sequence into the corresponding vector is successful. Commonly, vectors or plasmids will incorporate a selection maker such as B-galactosidase to identify properly ligated sequences.

Once ligated, the expression vector including the nucleic acid sequence encoding chondroitinase AC may be inserted into a host cell for production of a functional chondroitinase AC polypeptide. Insertion is typically referred to as transformation of the host cell. Thus, transformation of the host cell involves the genetic alteration of the host cell resulting from the uptake and expression of the foreign genetic material, namely the uptake of the expression vector, which includes the nucleic acid sequence encoding chondroitinase AC. A variety of methods are available for transforming host cells with expression vectors. Among these include electroporation, heat shock and chemical shock. Transformation methods are well known in the molecular biology arts and typically include the preparation or providing of competent cells, incubating the expression vector in the presence of a condition which encourages the uptake of the expression vector by the competent cells and selecting for cells that have taken up the expression vector. Often selection occurs by culturing cells in the presence of a selectable marker such as the use of an antibiotic (e.g. ampicillin, tetracycline, gentamycin and the like) to selectively propagate only those cells which express an antibiotic resistance gene, which is limited to host cells that have taken up and are capable of expressing sequences within the expression vector. Transformed cells are cultured until a desired amount is obtained. The number of cells may be determined using traditional cell counting techniques or using techniques such as measuring the absorbance of the culture at a suitable wavelength.

Expression of chondroitinase AC in a host cell may occur automatically by the host cell or may be induced by the user depending on the promoter. A variety of methods for inducing transcription of a nucleic acid sequence within an expression vector are known in the art. Thus the method of induction is intended to be nonlimiting as many methods may be used. For example, isopropyl-β-D-thio-galactoside (IPTG) is frequently used as an inducer of the lacZ operon. Briefly, IPTG binds to a lac repressor and inactivates it, thus allowing transcription to occur. The use of IPTG in recombinant expression systems is commonly used because it is not a substrate for β-galactosidase. Similarly the presence or absence of glucose may also be used for modulating transcription in a suitable system. The time of induction may vary depending on the efficiency of expression, the toxicity of the polypeptide within the cell, the efficiency of the expressed polypeptide and the like. Once induced, the sample may be cultured for hours, days and the like for the expression of chondroitinase AC and its cleavage of hyaluronic acid into hyaluronate unsaturated disaccharide. In one embodiment, the sample is cultured for approximately two days after induction begins.

Once expressed the exogenous chondroitinase AC polypeptide cleaves the endogenous hyaluronic acid. Cells should be cultured under conditions suitable for enzymatic activity. In some instances it may be preferred to change culture media prior to expression of the enzyme such as to remove an antibiotic. Enzymatic activity of chondroitinase AC was successful when incubated at about 37 degrees Celsius. Cleavage occurs at about 1-4 glycosidic bonds of hyaluronic acid. Preferably the hyaluronic acid is completely digested into hyaluronate unsaturated disaccharide. Chondroitinase AC may also cleave tetra- or hexa-saccharides into hyaluronate unsaturated disaccharide. Hyaluronate unsaturated disaccharide has a structure of D-glucuronic acid (GlcUA) (1-3)-D-N-acetyl-glucosamine (GlcNAc) and has a MW of 378.31.

The resulting hyaluronate unsaturated disaccharide is isolated from the culture. Isolation may include a variety of techniques used in the biochemical arts. Typically, the culture is spun down or centrifuged to separate the cells from the supernatant with the hyaluronate unsaturated disaccharide primarily found in the supernatant. Purification of hyaluronate unsaturated disaccharide from the supernatant may utilize one or more physical properties such as charge, size, hydrophobicity and the like.

In one embodiment, hyaluronate unsaturated disaccharide is purified through the use of affinity chromatography. Affinity chromatography is based on a highly specific biologic interaction such as between antigen and antibody, enzyme and substrate, or receptor and ligand. Affinity chromatography combines the size fractionation capability of gel permeation chromatography with the ability to design a stationary phase that reversibly binds to a known subset of molecules. Thus by providing a compound such as an antibody or antibody fragment against hyaluronate unsaturated disaccharide or binding partner, the desired disaccharide can be selectively removed from the supernatant. Often such methods are utilized by generating a column or solid phase bound to the antibody or binding partner, incubating the supernatant in the presence of the compound, removing the supernatant, washing the column or solid phase to remove unspecific interactions and eluting the bound disaccharide.

In another embodiment, the hyaluronate unsaturated disaccharide is purified from the supernatant using size exclusion chromatography. In size exclusion chromatography particles are separated based on their size, or in more technical terms, their hydrodynamic volume. It is usually applied to large molecules or macromolecular complexes such as proteins and industrial polymers. The underlying principle of size exclusion chromatography is that particles of different sizes will elute (filter) through a stationary phase at different rates. Provided that all the particles are loaded simultaneously or near simultaneously, particles of the same size should elute together. The molecular weight of the hyaluronate unsaturated disaccharide is about 378.31 and thus its elution can be determined in a particular system.

In another embodiment reversed-phase chromatography is used to purify hyaluronate unsaturated disaccharide from the supernatant. Reversed-phase chromatography refers to any chromatographic method that uses a non-polar stationary phase for separation. In essence, the introduction of alkyl groups covalently bonded to solid phase such as silica gel permits the elution of polar compounds before non-polar compounds. Any inert non-polar substance that achieves sufficient packing can be used for reversed-phase chromatography. C18 bonded silica and C8 bonded silica are commonly used in reversed-phase chromatography. Mixtures of water or aqueous buffers and organic solvents are used to elute compounds from a reversed phase column. The solvents should be miscible with water and the most common organic solvents used are acetonitrile, methanol or tetrahydrofuran (THF). Other solvents can be used such as ethanol, 2-propanol (isopropyl alcohol). Elution can be performed isocratic (the water-solvent composition does not change during the separation process) or by using a gradient (the water-solvent composition does change during the separation process). The pH of the mobile phase can have an important role on the retention of a compound and can change the selectivity certain compounds. Charged compounds can be separated on a reversed phase column by the use of ion-pairing (also called ion-interaction). This technique is known as reversed phase ion-pairing chromatography. In the preferred embodiment it was found that a C18 column washed with 50% acetonitril in water then 100% acetonitril, followed by elution with a 2:1 mixture of chloroform and methanol worked well. The presence of hyaluronate unsaturated disaccharide may be detected by measuring the absorbance at 240 nm.

The present invention may be adapted for any scale desired. Increasing the volume of culturing vessels such as wells, plates, flasks and the like may increase production of host cells and thus increase production of hyaluronate unsaturated disaccharide. Moreover by varying the density of cultures the use can modulate the amount of disaccharide produced. Thus, the volumes and conditions provided are for guidance and not strict requirements unless so indicated.

### EXAMPLES

The below provided examples are provided to illustrate and provide guidance to those skilled in the present art with respect to various embodiments encompassed within the present invention and are not intended to be limiting with respect to scope or technique used.

### Example 1

### Construction of an Expression Vector Including Chondroitinase Ac and its Transformation into a Host Cell

Synthesis of cDNA encoding chondroitinase AC (Chondroitin AC lyase). The double-stranded cDNA of chondroitinase AC was synthesized from the genomic DNA of Flavobacterium heparinum (ATCC#13125) purchased from American Type Culture Collection (ATCC, Manassas, Va.) by using Taq DNA polymerase and a set of upstream and downstream oligonucleotide primers for chondroitinase AC. By using Wizard Genomic DNA Purification Kit (Promega, Madison, Wis.), the genomic DNA was extracted from Flavobacterium heparinum induced for 32 hours at 23 degrees Celsius in an induction medium containing 1.0-1.5% chondroitine sulfate A. The primers included SEQ ID NO. 1 and SEQ ID NO. 2 which are provided in Table 1. The 5′ primer (AC-A) contained a XhoI site and the coding sequence for the first 7 amino acids from the chondroitinase AC. The 3′ primer (AC-B) contained a SalI site and coding sequence for the last 6 amino acids from the chondroitinase AC. The PCR buffer contained 50 mM KCl, 10 mM Tris-HCl (pH9.0). 1.5 mM MgCl.sub.2, 0.01% gelatin, 0.05 mmol each of dNTP, 1.0 umol of each primers, 10 ul reverse transcription reaction mixture, and 2 units of Taq DNA polymerase in a total of 50 ul volume. The PCR condition was 95 degrees Celsius for 60 seconds, 60 degrees Celsius for 60 seconds, and 72 degrees Celsius for 60 seconds for 30 cycles in the MJ Research model PTC-1152 thermocycler (MJ Research, Watertown, Mass.).

TABLE 1
Primers used in PCR to synthesize
chondroitinase AC

| SEQ ID NO: | Designation | Primer Sequence |
|---|---|---|
| SEQ ID NO: 1 | AC-A | 5′-ACTCGAGTCAGCAG |
| | | ACCGGTACTGCAGA-3′ |
| SEQ ID NO: 2 | AC-B | 5′-GTCGACTATTTCAG |
| | | TTCAACCGTTGCAC-3′ |

The PCR amplified DNA fragments were gel-purified and cloned into pGEM-T vectors (Promaga, Madison, Wis.). The PCR cDNA fragments with 3′ A overhangs can be directly ligated into pGEM-T cloning vectors without any digestion of restriction endonuclease. After ligation, the DNA was then transformed into competent E. coli DH5 alpha cells. Designing the restriction endonuclease sites in the primers can be used for subcloning the cDNA fragments into a variety of expression vectors.

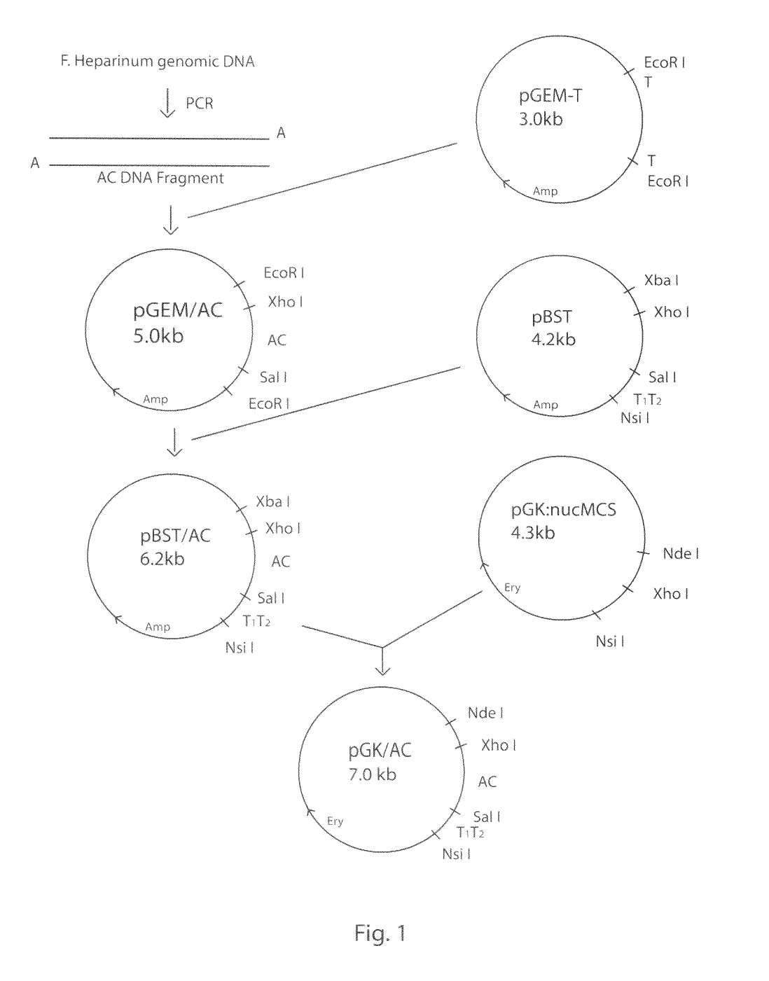

The plasmid isolated from one of the colonies was confirmed to contain the right size of the insert by the analyses of restriction endonucleases and by DNA sequence in both directions by the chain termination method (Sanger et al., Pro. Natl. Acad. Sci. 74:5463, 1977) to verify the DNA sequence of chondroitinase AC obtained was in agreement with the published nucleotide sequence of chondroitinase AC (Genbank with an accession #U27583) except at positions 1 to 6 (6 synthetic nucleotides for the clone purpose), at position 433 (thymidine instead of cytidine) and position 913 (thymidine instead of adenine) (SEQ ID NO: 3). Its corresponding amino acid sequence (SEQ ID NO: 4) shows that two amino acids extension, serine and threonine, at the amino terminus of chondroitinase AC, and the two arginines at the amino acid positions 145 and 305 were replaced by tryptophans respectively, in comparison to the published amino acid sequence of chondroitinase AC (Tkalec et al. Appl. Environ. Microbiol. 66:29, 2000). The plasmid containing the DNA insert encoding chondroitinase AC is designated as pGEM/AC (FIG. 1). Plasmid pGEM/AC was digested with restriction endonucleases XhoI and SalI to release the DNA insert encoding the chondroitinase AC. The DNA fragments were gel purified and then through the XhoI and SalI sites ligated to a slightly modified pBluescript II (SK+) cloning plasmid (Stratagene, La Jolla, Calif.) by the insert of rrnB operon DNA fragment carrying the sequence of rrnB T1T2 terminators, which is named here as pBST plasmid. After ligation, the DNA was then transformed into competent E. coli alpha-DH5 cells. The plasmid containing the DNA insert encoding chondroitinase AC is designated as pBST/AC (FIG. 1).

Construction of the Expression Vector pGK/AC. Plasmid pBST/AC was digested with restriction endonucleases XhoI and NsiI to release a DNA fragment carrying structural gene of Chondroitinase AC and terminator of rrnB T1T2. This DNA fragment was gel purified and then through the XhoI and NsiI sites ligated to a plasmid pGK:nucMCS (ATCC#87139) purchased from American Type Culture Collection. Manassas, Va., which has been described as a secretion reporter system in Gram-positive bacteria (Loir et al., J. Bacteriology 176:5135-5139.1994). After ligation, the DNA plasmid was transformed into naturally competent cells of streptococcus gordonii V288 (ATCC#35105). After the plasmid isolated from one of the colonies on THB/Em^{R} (Todd Hewitt Broth containing erythromycin at a final concentration at 10 microgram/ml) plates was confirmed by the analyses of restriction endonucleases and by DNA sequence to carry the chondroitinase AC and the rrnB T1T2 terminator. The plasmid is designated as pGK/AC. (see FIG. 1). Plasmid pGK/AC is a recombinant expression vector including DNA sequence provided in SEQ ID NO: 3 that encodes bacterial chondroitinase AC, provided in SEQ ID NO: 4.

Transformation of S. equi ss zooepidemicus. Preparation of S. equi ss zooepidemicus competent cells for transformation was provided ad follows. Specifically the present example describes the culturing of Streptococcus equi subsp. Zooepidemicus, ATCC#35246 however the present method may be applied to a variety of strains. This strain is a member of Lancefield's Group C. Brain Heart Infusion (BHI) broth (Difco 0037) was used to grow this cell at 37 degrees Celsius without shaking. TSA/SB (Trypticase Soy Agar with 5% Defibrinated Sheep Blood) plates were used to exhibit beta hemolysis of this strain.

Specifically S. equi ss zooepidemicus competent cells were prepared as follows. A 0.25 ml of over-night culture was diluted with 25 ml BHI broth and cultured at 35 degrees Celsius without shaking over-night (˜14 hours). One (1) ml of the culture was removed and dilute (1:100) with 100 ml THB (Todd Hewitt Broth) containing 0.6% glycine. Culturing at 35 degrees Celsius continued without shaking overnight (˜14 hours). The cells were further diluted 1:10 with THB containing 0.6% glycine and continue in culture at 35 degrees Celsius for 3.0 hours until OD600 reach between 0.2 and 0.3. The culture was chilled on ice for 15 minutes and the cells washed 3 times with cold 10% glycerol. The cells were resuspend with 7.5 ml cold 10% glycerol and aliquoted 100 microliter per tube and store at −70° C.

The competent cells were transformed as follows. Transformation of S. equi ss zooepidemicus was carried out by electroporation. 3 microgram pGK/AC plasmid DNA was added to 100 microliter of the cell suspension on ice and mixed and kept on ice for 5 minutes before electroporation. Electroporation was carried out in 0.1 cm cuvette at voltage 2500 V (25 kV/cm), resistance 600 ohms, and capacitance 25 μF in a BTX electroporator ECM 630 (Harvard Apparatus. MA). The result reading was 2447 V in voltage and 11.8 msec in the pulse length. The suspension was immediately placed on ice for 5 minutes and then diluted with 950 microliter BHI (Brain Heart Infusion) broth. The cell suspension was cultured at 37 degrees Celsius for 2 hours before plated on TSA/SB/Em^{R} (Trypticase soy agar with 5% defibrinated sheep blood containing erythromycin at a final concentration at 10 microgram/ml). The plates were cultured at 37 degrees Celsius for 38 hours. The colonies exhibiting beta hemolysis on TSA/SB/Em^{R} were picked and sub-cultured in 5 ml of BHI broth containing erythromycin at a final concentration at 10 microgram/ml at 35 degrees Celsius, 220 rpm shaking for 48 hours. 1.5 ml cells were spun down. The cell pellet was resuspended with 500 microliters TE buffer (50 mM Tris.HCl/10 mM EDTA, pH8.0) containing 30 mg lysozyme and 15 units of mutanolysin and incubated at 37 degrees Celsius for 60 minutes. The next steps were followed by the procedure indicated in the QIAprep Kit (Cat#27104, Qiagen, Germantown, Mass.). The analysis of restriction endonucleases indicated the correct DNA plasmid containing a correct DNA insert, which was further confirmed by DNA sequence in both directions by the chain termination method (Sanger et al., Pro. Natl. Acad. Sci. 74:5463, 1977). The cell clone is then named pGK/AC/Zooepidemicus.

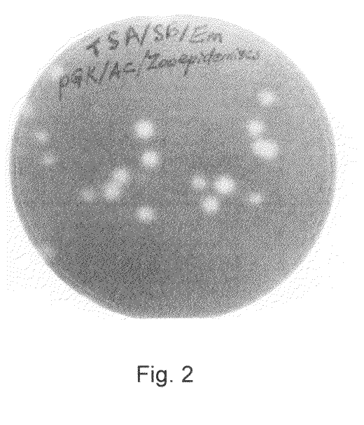

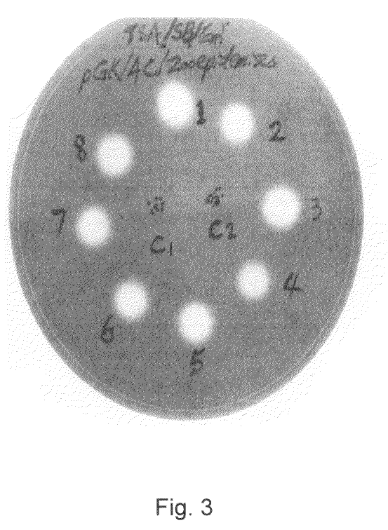

The verification of a cell clone containing pGK/AC may be seen in FIGS. 2 and 3. Referring to FIG. 2, the Beta-hemolysis around the colonies on TSA/SB/Em^{R} (Trypticase Soy Agar with 5% Defibrinated Sheep Blood containing erythromycin at a final concentration at 10 microgram/ml) was observed after the transformation of S. Zooepidemesis with plasmid pGK/AC by electroporation and culture at 37 degrees Celsius for 38 hours. Referring to FIG. 3, after inoculation with the colonies from TSA/SB/Em^{R}, the culture in THB/Em^{R} was incubated at 37 degrees Celsius and 220 rpm for 38 hours. 2 microliters of the sub-culture was plated on TSA/SB/Em^{R} (Trypticase Soy Agar with 5% Defibrinated Sheep Blood containing erythromycin at a final concentration at 10 microgram/ml). Beta hemolysis occurred around the cell culture after incubation at 37 degrees Celsius overnight (16 hours). The culture of S. equi ss zooepidemicus without transformation showed beta hemolysis on TSA/SB does not grow on TSA/SB/Em^{R} and shows no hemolysis (see wells C1 & C2).

### Example 2

### Production of Delta-HA Disaccharide

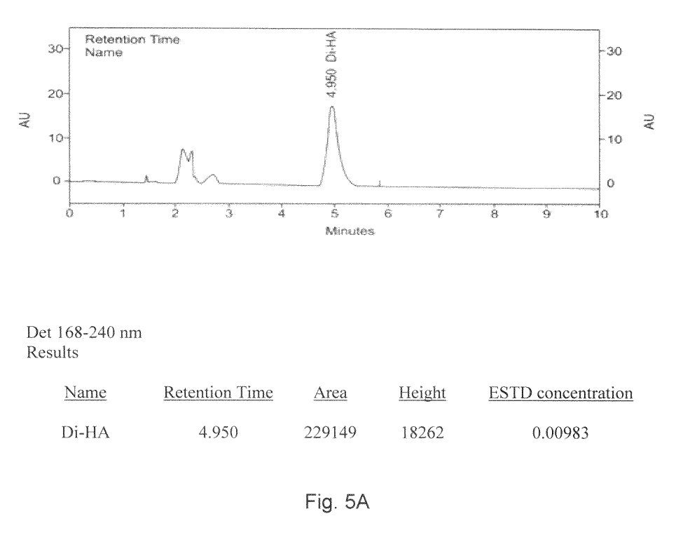

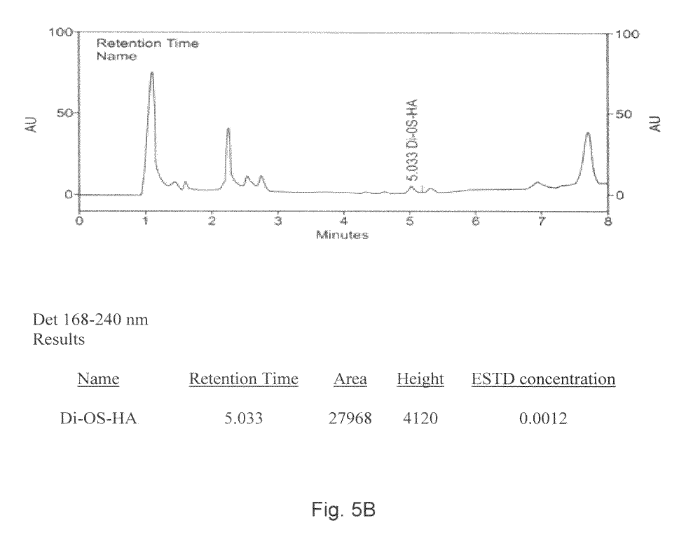

The cell clone pGK/AC/Zooepidemicus (as demonstrated in Example 1) was cultured in 15 ml THB/Em^{R} (Todd Hewitt Broth containing erythromycin at a final concentration at 10 microgram/ml) at 37 degrees Celsius overnight. After 1:10 dilution of the overnight culture with THB, the cell was cultured at 30 degrees Celsius without shaking for 2 days. A glucose solution and sodium acetate buffer, pH 6.2, was added to the culture to final concentrations of 1.6% glucose, and 100 mM sodium acetate buffer. The culture was continued at 37 degrees Celsius with shaking at 220 rpm for 2 days (48 hours). The cells were chilled for 5 minutes before spinning down. After 3500 rpm for 15 minutes, the supernatant was underwent Sep-Pak C18 purification. Briefly, a Sep-Pak Plus C18 column from Waters (Milford, Mass.) was equilibrant with 5 ml of mixture 2:1 (chloroform:methanol), then 5 ml acetonitril, followed by 5 ml of 50% acetonitril in H2O. After loading 20 ml supernatant, the column was washed with 10 ml 50% acetonitril in H2O, and followed by 10 ml 100% acetonitril. The delta-HA disaccharide was eluted from the column with 5 ml of 2:1 mixture of chloroform and methanol. The eluate was then dried by speed-vac at 55 degrees Celsius. The dried pellet was re-suspended in 300 microliters of H2O. The delta-HA disaccharide was separated by RP-HPLC and detected by UV 240 nm (see FIG. 5). The production of the delta-HA disaccharide was about 0.001 mg/ml.

Table 2 depicts a summary of unsaturated chondroitine sulfate disaccharides. The measurements were based on the production of unsaturated chondroitine sulfate disaccharides exhibiting a remarkable UV absorbance at 230 nm by an enzymatic reaction. A typical incubation mixture contained 270 microliters of culture supernatant, 15 microliters chondroitin sulfate A (substrate). 50 mM Tri-HCl buffer solution (pH 8.2) containing 50 mM sodium acetate in a total volume of 300 microliters volume. After incubation at 37 degrees Celsius overnight, the reaction was stopped by heating for 1 min in a boiling water bath. The reaction mixture was then diluted with 100 volumes of water, and UV absorption of the resultant solution was measured at 232 nm against the corresponding control mixture. A negative control mixture, a heat-inactivated supernatant or chondroitinase ABC solution, or a positive control (0.02 units of chondroitinase ABC), was kept in a substrate solution with the same composition as above and treated in the same manner as above. A cell culture of S ss. Zooepidemicus was used as a second control. The cell clone of pGK/AC/Zooepidemises shows enzymatic activities of chondroitinase AC in their supernatants.

TABLE 2

| Sample No. | Boiled (UV = 232 nm) | Un-boiled (UV = 232 nm) |
|---|---|---|
| Clone #1 | 0.501 | 0.513 |
| Clone #2 | 0.490 | 0.503 |
| Clone #3 | 0.494 | 0.505 |
| Clone #4 | 0.497 | 0.520 |

The dye-binding assay of Hotez et al was used as a second method to determine whether AC is present in the clone supernatants. Results are shown in Table 3 below. Specifically, the supernatant (80 microliters) was incubated with 10 micrograms of hyaluronic acid in a sodium acetate buffer pH 6.2, in a total volume of 100 microliters. The reaction was then incubated at 37 degrees Celsius for 24 hours and stopped by the addition of 900 microliters of Stain-All solution (17 mg Stains-All, 50% formamide. 0.06% glacial acetic acid in a final volume of 100 ml). The absorbance was then immediately read at 640 nm. HA, but not its products of degradation interacts with the carbocyanine dye Stain-All to shift the wavelength of maximal absorbance in the visible spectrum of the dye toward longer wavelength. Separately, a heat denatured supernatant, as a control was kept in a substrate solution with the same composition as above and treated in the same manner as above to measure the absorbance at 640 nm. The results indicate that the supernatant and the cell lysate of pGK/AC/Zooepidemises contain chondroitinase AC activities.

TABLE 3

| | Boiled (UV = 640 nm) | Un-boiled (UV = 640 nm) |
|---|---|---|
| Sample No. | (Supernatant/Lysate) | (Supernatant/Lysate) |
| Clone #1 | 2.323/2.491 | 2.317/2.384 |
| Clone #2 | 2.668/2.519 | 2.408/2.430 |
| Clone #3 | 2.476/2.648 | 2.353/2.628 |
| Clone #4 | 2.509/2.693 | 2.434/2.609 |

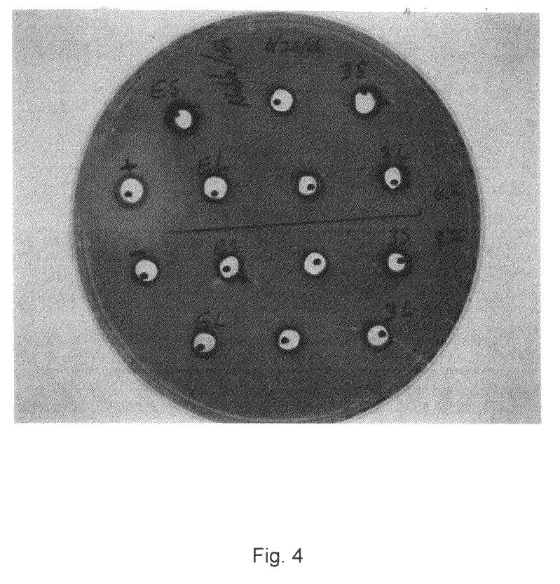

A Nobel-HA (hyaluronic acid)/Nobel-CS (chondroitine sulfate) Plates Assay was performed to detect the presence of AC. The results are depicted in FIG. 4. Specifically, the Nobel-HA plate contained 1% Noble agar 400 mcg/ml HA, and 1% bovine serum albumin and the Nobel-CS plate contained 1% Noble agar, 1200 mcg/ml CS, and 1% bovine serum albumin. Holes were punched into the agar and 80 microliters of supernatant from the cell culture containing 100 mM sodium acetate buffer, pH6.2 was placed in each well of the Nobel-HA plate. 80 microliters of supernatant from the cell culture containing 100 mM sodium acetate buffer, pH8.2 was placed in each well of the Nobel-CS plate. A positive control included chondroitinase ABC 0.04 unites, whereas non-tranformated cell culture served as a negative control. The plate was then reversed, sealed, and incubated at 37 degrees Celsius. After 3 days of incubation at 37 degrees Celsius, the plates were incubated with 50% formamide for 30 minutes, then with Stain-All solution for 2 hours at room temperature wrapped with foil to avoid the light. The plates were then washed with water. A zone of clearing around the well 3S (pH 6.2) or ES (pH 8.2) can be seen indicating the presence of chondroitinase AC.

What is claimed is:

1. A method of producing a hyaluronate unsaturated disaccharide comprising:
a) providing a host cell capable of expressing a native hyaluronic acid, said host cell comprising a recombinant expression vector comprising a nucleic acid sequence encoding a chondroitinase AC polypeptide operably linked to a promoter, wherein said host cell does not express a native chondroitinase AC polypeptide;
b) culturing said host cell in conditions suitable for expression of said chondroitinase AC polypeptide and for a time sufficient for digestion of hyaluronic acid into hyaluronate unsaturated disaccharide by expressed chondroitinase AC polypeptide; and
c) isolating said hyaluronate unsaturated disaccharide.

2. The method according to claim 1, wherein said host cell is a Group C streptococci or S. equi ss zooepidemicus.

3. The method according to claim 1, wherein said nucleic acid sequence is a cDNA nucleic acid sequence or a genomic DNA sequence.

4. The method according to claim 1, wherein said nucleic acid sequence comprises the nucleic acid sequence of SEQ ID NO.: 3.

5. The method according to claim 1, wherein said chondroitinase AC polypeptide comprises the amino acid sequence of SEQ. ID NO.: 4.

6. The method according to claim 1, wherein said isolation comprises purifying said hyaluronate unsaturated disaccharide by a method selected from the group consisting of affinity chromatography, size exclusion chromatography and reversed-phase chromatography.

7. The method according to claim 1, wherein greater than 1 microgram of hyaluronate unsaturated disaccharide is isolated.

8. The method according to claim 1, wherein greater than 1 milligram of hyaluronate unsaturated disaccharide is isolated.

9. A method of producing a hyaluronate unsaturated disaccharide comprising:
a) providing a host cell capable of expressing a native hyaluronic acid, said host cell comprising a recombinant expression vector comprising a nucleic acid sequence encoding a chondroitinase AC polypeptide operably linked to a promoter;
b) inducing expression of said chondroitinase AC polypeptide;
c) incubating said host cell in conditions suitable for digestion of hyaluronic acid into hyaluronate unsaturated disaccharide by said chondroitinase AC polypeptide; and
d) isolating said hyaluronate unsaturated disaccharide.

10. The method according to claim 9, wherein said nucleic acid sequence is a cDNA nucleic acid sequence or a genomic DNA sequence.

11. The method according to claim 9, wherein said nucleic acid sequence comprises the nucleic acid sequence of SEQ ID NO.: 3.

12. The method according to claim 9, wherein said chondroitinase AC polypeptide comprises the amino acid sequence of SEQ. ID NO.: 4.

13. A host cell capable of expressing native hyaluronic acid transfected or transformed with a recombinant expression vector comprising a nucleic acid sequence encoding a chondroitinase AC polypeptide operably linked to a promoter.

14. The host cell according to claim 13, wherein said host cell is a Group C streptococci or S. equi ss zooepidemicus.

15. The host cell according to claim 13, wherein said host cell does not express a native chondroitinase AC polypeptide.

16. The host cell according to claim 13, wherein said nucleic acid sequence comprises the nucleic acid sequence of SEQ ID NO.: 3.

17. The method according to claim 13, wherein said chondroitinase AC polypeptide comprises the amino acid sequence of SEQ. ID NO.: 4.

18. A method of producing a hyaluronate unsaturated disaccharide comprising:
a) providing a host cell capable of expressing a native hyaluronic acid,
b) transforming said host cell with a recombinant expression vector comprising a nucleic acid sequence encoding a chondroitinase AC polypeptide operably linked to a promoter;
c) inducing expression of said chondroitinase AC polypeptide;
d) incubating said host cell in conditions suitable for digestion of hyaluronic acid into hyaluronate unsaturated disaccharide by said chondroitinase AC polypeptide; and
e) isolating said hyaluronate unsaturated disaccharide.
